(19) United States
(12) Patent Application Publication
(10) Pub. No.: US 2020/0281810 A1
(43) Pub. Date: Sep. 10, 2020

(54) SKULL VIBRATION DEVICE AND METHOD FOR USING THE SAME

(71) Applicant: NETEN INC., Kofu-shi (JP)

(72) Inventors: Kenji NANASAWA, Kofu-shi (JP); Tomoki NANASAWA, Kofu-shi (JP); Masayuki MATSUBAYASHI, Kofu-shi (JP)

(73) Assignee: NETEN INC., Kofu-shi (JP)

(21) Appl. No.: 16/810,015

(22) Filed: Mar. 5, 2020

(30) Foreign Application Priority Data
Mar. 7, 2019 (JP) 2019-041976
Aug. 29, 2019 (JP) 2019-157346

(51) Int. Cl. A61H 23/02; A61L 31/02

(52) U.S. Cl. CPC A61H 23/0245; A61L 31/022; A61H 2205/02; H03F 3/04; A61H 2201/0119; A61H 2203/0425; A61H 2203/0406; A61H 2209/00

(57) ABSTRACT

A skull vibration device includes: an electrical signal generator configured to generate an electrical signal; an amplifier configured to amplify the electrical signal from the electrical signal generator; a vibration generator configured to convert the electrical signal transmitted from the amplifier to a mechanical vibration; a metal vibration member to which a vibration generating portion of the vibration generator is directly coupled, and which is configured to cover a skull of a human body with a predetermined spacing left between the vibration member and the skull; and a vibration member supporter configured to suspend the vibration member while preventing direct or indirect contact of the vibration member with the human body, and to keep the vibration member at a predetermined distance from the skull. A vibration generated from the vibration member is transferred to the skull through a vibration of air.

### CROSS-REFERENCE TO RELATED APPLICATION

This application claims priority to Japanese Patent Application No. 2019-041976 filed on Mar. 7, 2019 and Japanese Patent Application No. 2019-157346 filed on Aug. 29, 2019, the disclosures of which including the specifications, the drawings, and the claims are hereby incorporated by reference in their entireties.

### BACKGROUND

A treatment called a cranial technique (craniosacrum therapy) has been known to date. A cranium (cranio) means a “skull” and the cranial technique is employed to relieve a strain and/or a tension of the skull and unstiffen a hardened portion of the body. A skull repeatedly expands and shrinks several times, for example, five times, in one minute, to thereby send cerebrospinal fluid to the whole body, that is, perform a breath-like operation (also called a primary breath). If the skull is locked in this operation, cerebrospinal fluid cannot flow smoothly. In view of this, the cranial technique is employed to adjust the skull to adjust cerebrospinal fluid and cause the fluid to flow smoothly so that immunocompetence is enhanced and a person's health is improved in whole body.

The skull is supported by backbones, and the sacrum is present at the bottom of the backbones. The structure from the skull to the sacrum is common to all the vertebrates including human. The neural network of the total body is built around the spines, and the balance of the body is achieved by the column constituted by the skull, the spinal cord, and the sacrum.

For this reason, there has been an idea of improving health by adjusting these parts. For example, in the field of healing, chakras correspond to these parts, and the first to seventh chakras are sequentially allocated to the sacrum, the backbones, and the skull. Thus, it is very important to adjust these parts.

As described in Japanese Unexamined Patent Application Publication No. 2011-160869, there has been a sound therapy bed including: a bed body having a baffle board at the upper surface thereof; a sound hole provided in the baffle board; and a speaker disposed below the sound hole with the baffle board interposed therebetween and inside the bed body. The sound therapy bed ensures transfer of sound waves and vibration waves necessary for a sound therapy to a human body and enhances therapy effects.

There has also been a headphone that vibrates the skull and transfers the vibrations directly to the tympanic membranes so that the user recognizes the vibrations as sound.

Japanese Patent No. 5517087 proposes a tuning fork-type treatment device that transfers vibrations to the upper jaw and the lower jaw through teeth, causes the zygomatic bones and the skull to vibrate, and applies a vibration stimulus directly to the human brain by bone conduction.

In a cranial therapy, the skull is generally touched gently and is moved under a subtle pressure.

Although there has been a need to apply a stimulus indirectly without directly applying bone vibrations to the skull, conventional vibration devices basically directly vibrates the skull. This is because backbones and other bones need a certain degree of stimulus in many cases.

On the other hand, a therapy using tuning forks as proposed in Japanese Patent No. 5517087 is conceivable. It is however difficult to conduct such a therapy by one person, and preparation of a plurality of types of tuning forks is also difficult.

Among bones constituting the skull, the sphenoid bone is located ahead of the bottom of the occipital bone and a front portion of the sphenoid bone reaches the nasal cavities. There is also a need to relax this portion for enhancing the health.

It is therefore an object of the present disclosure to appropriately vibrate a skull with a simple method.

### SUMMARY

To achieve the object, according to this disclosure, a vibration member covering a skull with a predetermined spacing left between the vibration member and the skull is caused to vibrate.

Specifically, in a first aspect of the disclosure, a skull vibration device includes: an electrical signal generator configured to generate an electrical signal; an amplifier configured to amplify the electrical signal from the electrical signal generator; a vibration generator configured to convert the electrical signal transmitted from the amplifier to a mechanical vibration; a metal vibration member to which a vibration generating portion of the vibration generator is directly coupled, and which is configured to cover a skull of a human body with a predetermined spacing left between the vibration member and the skull; and a vibration member supporter configured to suspend the vibration member while preventing direct or indirect contact of the vibration member with the human body, and to keep the vibration member at a predetermined distance from the skull, wherein a vibration generated from the vibration member is transferred to the skull through a vibration of air.

With this configuration, the skull is vibrated not by contact of the vibration member with the head but by vibrations of air using the metal vibration member covering the skull with a predetermined spacing left therebetween. Thus, the skull can be appropriately vibrated with efficiency by using suitable vibrations. In addition, it is sufficient to select and transmit an electrical signal suitable for vibrating the skull from the electrical signal generator. Thus, it is unnecessary to prepare a large number of tuning forks, unlike a tuning fork-type treatment device.

In a second aspect, in the skull vibration device of the first aspect, the vibration member may be made of a titanium semispherical plate member.

With this configuration, since titanium has an internal attenuation factor with respect to sound, undulations generated by vibrations can be easily transferred to other media without being absorbed in titanium itself. In addition, since the vibration member is made of the semispherical plate, an appropriate distance can be kept from the skull. Accordingly, the skull can be appropriately vibrated with higher efficiency. The “semispherical” herein includes not only a perfect semispherical shape having a circular cross section, but also a dome shape and a cap shape each of which is an elongated circle or an oval in cross section, in a broad sense.

In a third aspect, in the skull vibration device of the second aspect, the vibration member may be made of a crystallized titanium semispherical plate member subjected to anodic oxidation.

With this configuration, the titanium member subjected to anodic oxidation (decorative crystallization processing) noticeably exhibits advantages unique to titanium so that the skull can be appropriately vibrated with higher efficiency.

In a fourth aspect, in the skull vibration device of any one of the first to third aspects, the vibration member may have a through hole for suspension.

With this configuration, the use of the vibration member in a suspended state makes it possible to vibrate the skull appropriately with efficiency while hindering transfer of vibrations to other members.

In a fifth aspect, in the skull vibration device of any one of the first to fourth aspects, the vibration generator may be constituted by one or more piezoelectric devices attached to the vibration member.

With this configuration, the skull can be appropriately vibrated with higher efficiency using thin piezoelectric devices that are available at low costs. The piezoelectric device is also called a piezoelectric element, and is a passive element that converts a voltage to a force and uses an inverse piezoelectric effect. The piezoelectric device here is suitable for generating appropriate vibrations. The number of the piezoelectric devices and locations of attachment of the piezoelectric devices are not specifically limited.

In a sixth aspect, a method for using a bone vibration device is provided. The bone vibration device includes an electrical signal generator configured to generate an electrical signal, an amplifier configured to amplify the electrical signal from the electrical signal generator, a vibration generator configured to convert the electrical signal transmitted from the amplifier to a mechanical vibration, a metal vibration member to which a vibration generating portion of the vibration generator is directly coupled, and which is configured to cover a skull of a human body with a predetermined spacing left between the vibration member and the skull, and a vibration member supporter configured to suspend the vibration member while preventing direct or indirect contact of the vibration member with the human body, and to keep the vibration member with the predetermined spacing left between the vibration member and the skull. The method includes the steps of: hanging the skull vibration device from a ceiling or a suspension member; allowing a person to stand or sit while a skull of the person is located inside the vibration member with a predetermined spacing left between the skull and the vibration member; and transmitting an electrical signal from the electrical signal generator and causing the vibration generator to generate vibrations including a frequency at which the skull vibrates to thereby vibrate the skull.

With this method, it is possible to vibrate the skull by one person for a predetermined time using vibrations of air without asking a third party to play tuning forks, unlike a tuning fork-type treatment device. Accordingly, the skull can be appropriately vibrated with efficiency with appropriate vibrations.

In a seventh aspect, in the method of the sixth aspect, the bone vibration device may further include a seat electrical signal generator configured to generate an electrical signal, a seat amplifier configured to amplify the electrical signal from the seat electrical signal generator, a seat vibration generator configured to convert the electrical signal transmitted from the seat amplifier to a mechanical vibration, a metal seat vibration member to which a vibration generating portion of the seat vibration generator is directly coupled, and which includes a seat portion configured to contact the human body and a side portion continuous to the seat portion, and a seat supporting member disposed at a back side of the seat portion to cover the seat vibration generator and placed on a mount surface to support the seat portion with the seat portion separated from the mount surface such that transfer of vibration energy to the mount surface is reduced, and the method further includes the step of allowing the person to stand or sit with the skull of the person located inside the seat vibration member with the predetermined spacing left between the seat vibration member and the skull in a state where the person stands or sits on a bone vibration sensing device configured to receive a vibration from the human body that is in contact with the seat portion.

In this method combined with the bone vibration sensing device in which the vibration generator vibrates the seat portion of the metal vibration member supported in a free state and separated from the mount surface by using the seat supporting member so as to suppress transfer of vibration energy to the mount surface so that the human body is directly vibrated by bone conduction, vibrations can be transferred to the skull from the top and the bottom through moisture in the whole body, and heel bones or the sacrum and pelves. Accordingly, the skull can be appropriately vibrated with higher efficiency using more suitable vibrations.

In an eight aspect, the method of the sixth or seventh aspect may further include the steps of: preparing a pyramid component constituted by a metal frame as the suspension member; connecting another vibration generator in the suspension member to the amplifier; and allowing the person to stand or sit under the suspension member in a state where the vibration generator in the suspension member is vibrated.

With this method, the vibration generator disposed on the vibration member supporter constituted by the pyramid metal frame is also vibrated so that resonance occurs in the metal frame and the skull and the whole body can be appropriately vibrated more effectively. The vibration generator may be a piezoelectric device or a vibrotransducer, for example. The location on the metal frame to which the vibration generator is attached is not specifically limited.

As described above, according to the present disclosure, the vibration device is kept with a predetermined spacing left between the vibration device and the skull, and vibrations generated from the vibration member is transferred to the skull through vibrations of air. Accordingly, the skull can be appropriately vibrated by a simple method.

### BRIEF DESCRIPTION OF THE DRAWINGS

FIG. 1 is an enlarged perspective view illustrating a suspended skull vibration device.

FIG. 2 is a perspective view illustrating a state where a user uses the skull vibration device while the user is sitting.

FIG. 3 is a perspective view illustrating a state where a user uses the skull vibration device while the user is standing.

FIG. 4 is a plan view illustrating a vibration plate and a vibration generator.

FIG. 5 is a plan view illustrating the vibration plate.

FIG. 6 is a bottom view illustrating a vibration plate and a vibration generator according to a variation of an embodiment.

FIG. 7 is an overview illustrating an example of a usage state of a skull vibration device according to the variation of the embodiment.

### DETAILED DESCRIPTION

An embodiment will be described hereinafter with reference to the drawings.

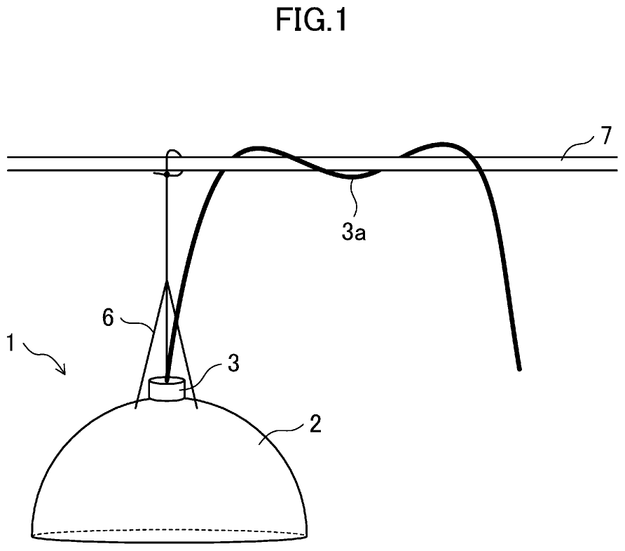

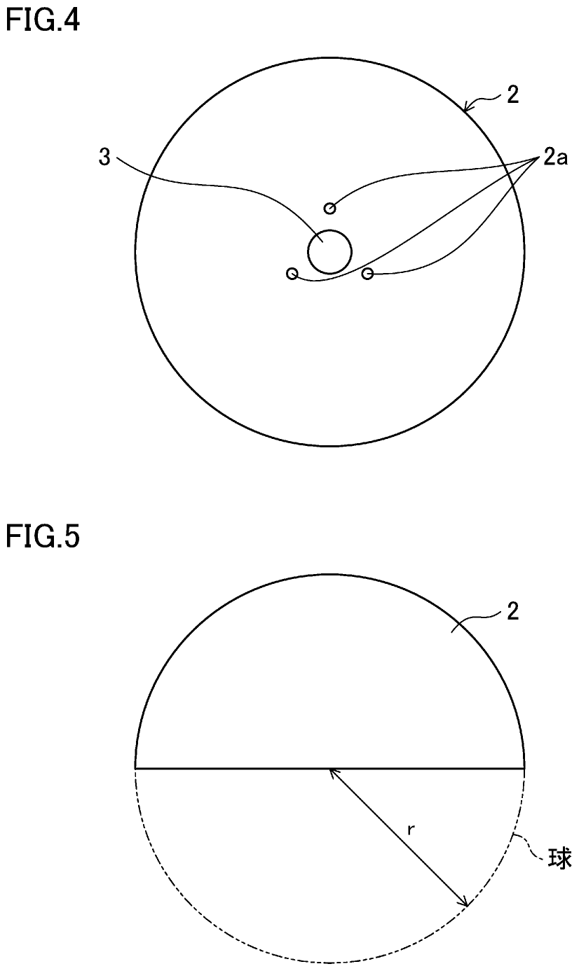

FIGS. 1 through 3 illustrate a skull vibration device 1 according to this embodiment. As also illustrated in FIGS. 4 and 5, the skull vibration device 1 includes a metal vibration member 2 having an inverted bowl-like shape. The vibration member 2 is made of a crystallized titanium semispherical plate subjected to decorative crystallization processing, for example. The decorative crystallization processing refers to a technique with which an anodic oxidation coating is formed by anode oxidation on a titanium surface and an uneven structure of the anodic oxidation coating reflects lightwaves, and only a specific frequency band is emphasized and is seen as a color. Titanium has an internal attenuation factor with respect to sound, and easily transfers undulations generated by vibrations to other media without absorbing the undulations in titanium itself. The semispherical shape of the plate can maintain an appropriate distance from a skull 51. Thus, the skull 51 can be vibrated appropriately with higher efficiency. A measurement of acoustic properties of a titanium rod subjected to decorative crystallization processing shows more excellent acoustic properties than general titanium. For example, the vibration member 2 is constituted by a semispherical plate member having a radius r of 225 mm such that the entire skull 51 of a common adult male is covered by the vibration member 2 with a spacing left at a predetermined distance (depending on the size of the skull 51) from the vibration member 2. The vibration member 2 has a thickness of about 1 mm, for example. The vibration member 2 is not limited to this size, and may be constituted by a metal thin plate of a material except for titanium.

Three through holes 2a, for example, are formed in a top portion of the vibration member 2. The through holes 2a are used for suspending the vibration member 2. A cord 6 or the like that does not easily transmit vibrations is used for the through holes 2a. As illustrated in FIG. 1, the cord 6 or the like is hooked on a pole 7 extending horizontally and/or a suspension hook 9 disposed on a ceiling 8, for example. The pole 7 and the suspension hook 9, for example, serve as vibration member supporters that keep the vibration member 2 at a predetermined distance from the skull 51.

One vibration generator 3, for example, is fixed to the top portion of the vibration member 2. The vibration generator 3 is constituted by a high-performance vibrotransducer capable of reproducing a frequency band of, for example, 20 Hz to 15,000 Hz. A vibration generating portion (not shown) of the bottom surface of the vibration generator 3 is directly coupled to the top portion of the vibration member 2 with an adhesive or the like so that an electrical signal transmitted from an amplifier 5 described later can be converted to mechanical vibrations.

The skull vibration device 1 includes an electrical signal generator 4 that generates an electrical signal. The electrical signal generator 4 is not specifically limited to a specific frequency generator capable of emitting specific low frequencies of compressional waves based on, for example, language frequencies, a CD player, a smartphone, and so forth.

The skull vibration device 1 includes the amplifier 5 that amplifies an electrical signal from the electrical signal generator 4. The amplifier 5 itself may incorporate the electrical signal generator 4 constituted by a player capable of reproducing, for example, an SD card.

Accordingly, vibrations generated from the vibration member 2 are transferred to the skull 51 through vibrations of air.

A method for using the skull vibration device 1 according to this embodiment will be described.

First, the electrical signal generator 4, the amplifier 5, and the vibration generator 3 described above, for example, are connected to an unillustrated power supply. In the case of using a bone vibration sensing device 101 at the same time, the bone vibration sensing device 101 is also connected to the power supply.

Although not described in detail, the bone vibration sensing device 101 is directly coupled to a vibration generating portion of a seat vibration generator 103 similar to the vibration generating portion described above, and includes a seat vibration member 102 and a seat supporting member 104. The seat vibration member 102 is made of a metal (e.g., titanium thin plate) and includes a seat portion 102a that contacts a human body 50 and a side portion 102b continuous to the seat portion 102a. The seat supporting member 104 is disposed at the back side of the seat portion 102a to cover the seat vibration generator 103, and supports the seat portion 102a in a state separated from a mount surface 108 such that the seat portion 102a is not placed on the mount surface 108 and transfer of vibration energy to the mount surface 108 is reduced. An incorporated seat amplifier 105 may be connected to the electrical signal generator 4 or another seat signal generator 106. A signal generated by the seat amplifier 5 is transferred to the seat vibration generator 103. With the bone vibration sensing device 101, the vibration generator vibrates the seat portion 102a of the seat vibration member 102 supported by the seat supporting member 104 in a free state separated from the mount surface 108 so that the human body can be vibrated directly by bone conduction. Accordingly, specific frequencies can be transferred to the human body as accurate as possible.

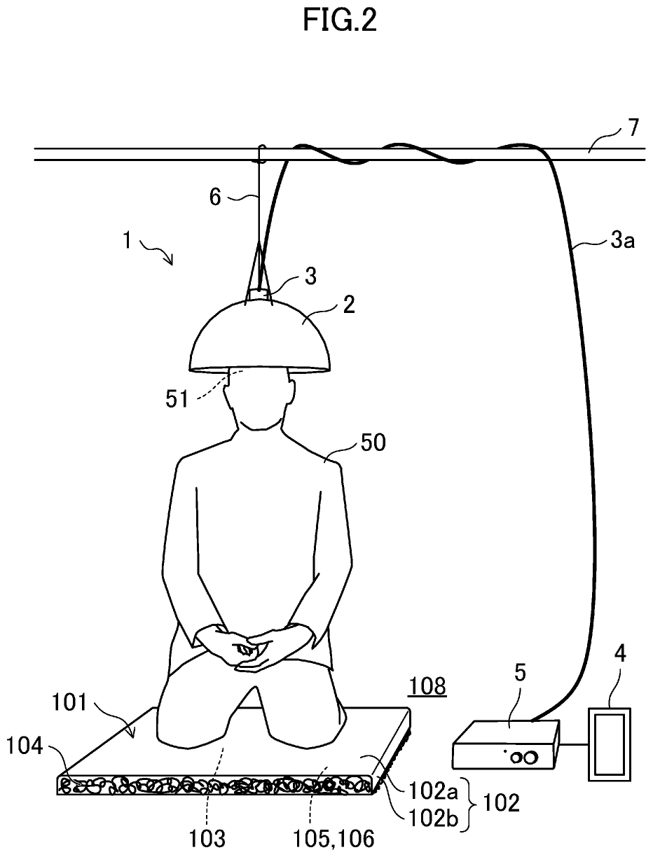

As illustrated in FIG. 2, the user sits straight or cross-legged on the bone vibration sensing device 101. In this state, the cord 6 penetrating the through holes 2a is tied to the pole 7 with the skull 51 kept inside the vibration member 2 at a predetermined distance from the vibration member 2. The length of the cord 6 is adjusted in accordance with the position of the human body 50. An electric wire 3a of the vibration generator 3 is suitably wound around the pole 7 and then connected to the amplifier 5. The amplifiers 5 and 105 may be connected to the electrical signal generator 4, or SD cards may be inserted in players incorporated in the amplifiers 5 and 105.

While the user sits on the bone vibration sensing device 101, electrical signals are sent from the amplifiers 5 and 105 to vibrate the vibration generator 3 and the seat vibration generator 103.

The electrical signal is not specifically limited, and an electrical signal that generates frequencies including, for example, 4000 Hz and 8000 Hz at which resonance (oscillation) easily occurs in the parietal bone and the occipital bone is selected. An electrical signal including an electrical signal that generates specific low frequencies of compressional waves of, for example, 6 Hz to 50 Hz may be emitted.

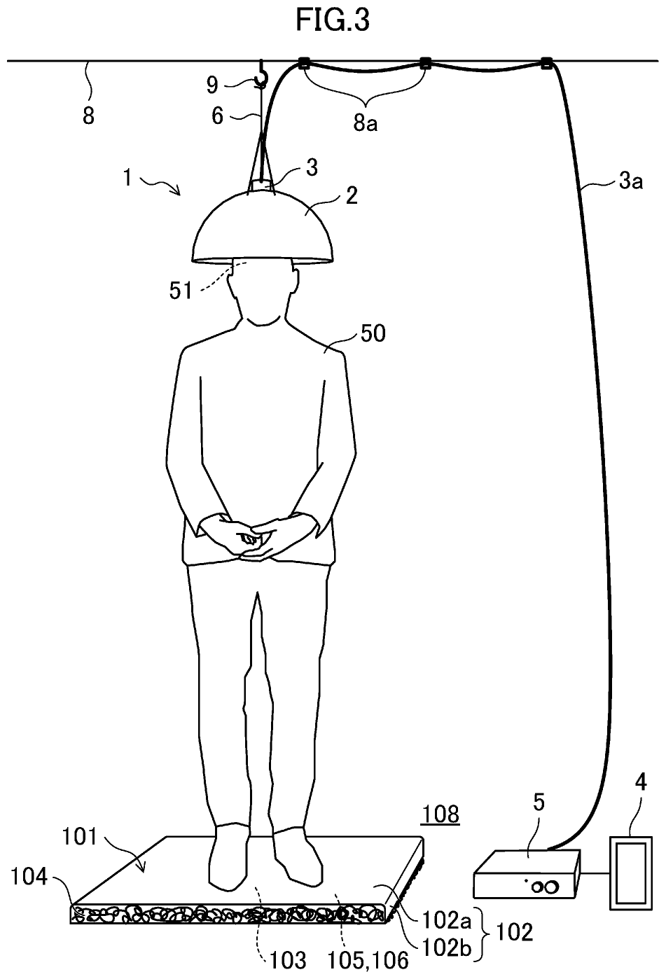

Similarly, as illustrated in FIG. 3, the user may use the skull vibration device 1 while the user stands. For example, the cord 6 is wound around the suspension hook 9 disposed on the ceiling 8, and the electric wire 3a of the vibration generator 3 is fixed to fasteners 8a disposed on the ceiling 8 at appropriate intervals and is connected to the amplifier 5. In this case, the length of the cord 6 is also adjusted in accordance with the height of the human body 50.

Then, an electrical signal is transmitted from the electrical signal generator 4 so that the vibration generator 3 generates vibrations including frequencies at which the skull 51 vibrates, and thereby, the skull 51 vibrates. While the user is standing on the bone vibration sensing device 101, an electrical signal is sent from the amplifier 105 so that the seat vibration generator 103 is vibrated.

In this manner, the skull 51 vibrates not by contact of the vibration member 2 with the head but by vibrations of air. Thus, the skull 51 can be vibrated appropriately with efficiency by using suitable vibrations.

In addition, it is possible to vibrate the skull 51 by one user for a predetermined time by a simple method using air vibrations without asking a third party to play tuning forks, unlike a tuning fork treatment device.

In addition, since the vibration member 2 is constituted by a crystallized titanium semispherical plate subjected to decorative crystallization processing, properties unique to titanium can be noticeably exhibited, and it is possible to vibrate the skull 51 appropriately with higher efficiency.

Furthermore, since the vibration member 2 is suspended from the pole 7 and the ceiling 8 through a material that does not easily transfer vibrations, such as the cord 6, it is possible to vibrate the skull 51 appropriately with efficiency while hindering transfer of vibrations to other members.

Moreover, with the combination with the bone vibration sensing device 101 in which the vibration generator 3 vibrates the metal seat portion 102a of the vibration member 102 supported in a free state and separated from the mount surface 108 by using the seat supporting member 104 so as to suppress transfer of vibration energy to the mount surface so that the human body 50 is directly vibrated by bone conduction, vibrations can be transferred to the skull 51 from the top and the bottom through moisture in the whole body, and heel bones or the sacrum and pelves. Accordingly, the skull 51 can be appropriately vibrated with higher efficiency with more suitable vibrations. The device may be used in such a manner that vibrations with some strength are applied from the bottom and an appropriate degree of vibrations is applied from the skull.

There is also an advantage that the whole body returns to an optimum condition by vibrating the sacrum and the sphenoid bone at the same time to shrink and/or loose the sphenoid bone to an appropriate position. Based on the idea that the sacrum and the sphenoid bone transfer their vibrations to each other through backbones, applications of vibrations from the top and the bottom have various advantages.

Thus, the skull vibration device 1 according to this embodiment can vibrate the skull 51 appropriately by a simple method.

By vibrating the skull 51 appropriately, advantages similar to those of a cranial technique can be obtained, and cerebrospinal fluid is adjusted to flow smoothly by adjusting the skull so that immunocompetence is enhanced and a person's health can be improved in whole body.

### —Variation—

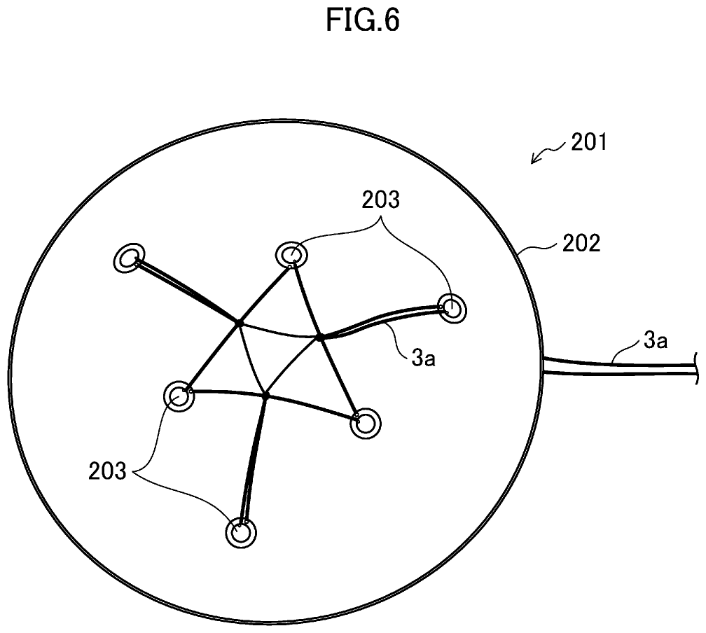

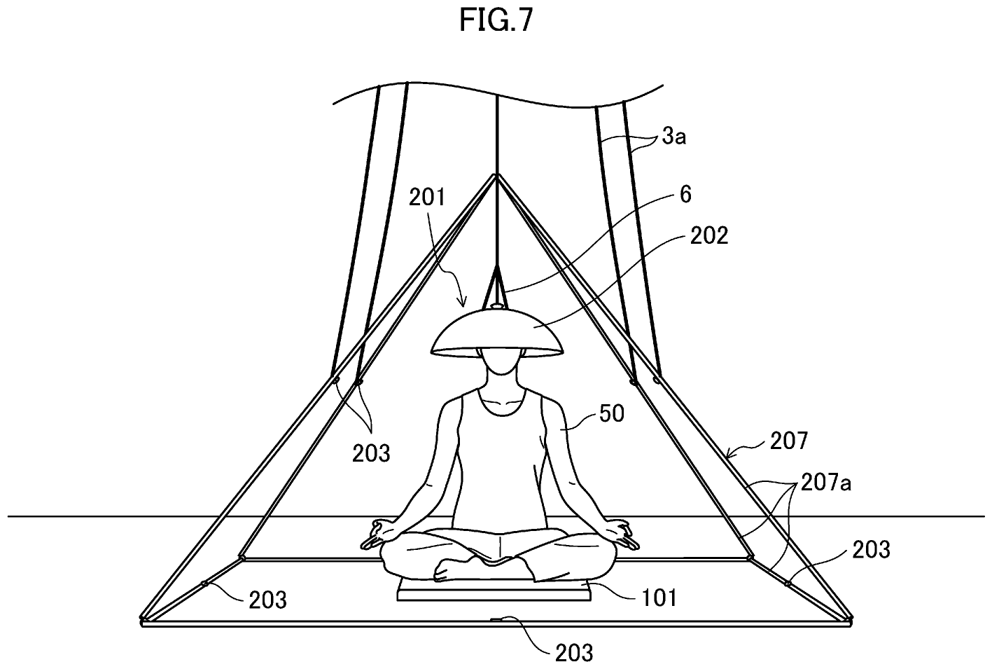

FIGS. 6 and 7 illustrate a skull vibration device 201 according to a variation of the embodiment. The skull vibration device 201 is different from the skull vibration device 1 of the embodiment in vibration generators 203. In this variation, components of the configuration already described with reference to FIGS. 1 through 5 are denoted by the same reference numerals, and description thereof will not be repeated.

A skull vibration device 201 according to this variation uses piezoelectric devices as the vibration generators 203. The piezoelectric devices have advantages of small thickness for small installation space, and availability at low costs.

FIG. 6 illustrates an example in which the vibration generators 203 are attached to the inner surface of the vibration member 202. Locations of attachment of the vibration generators 203 and the number of the vibration generators 203 are not limited to those of this example, and the vibration generators 203 may be attached to the outer surface of the vibration member 202.

As illustrated in FIG. 7, the shape of the vibration member 202 according to this variation is similar to a round cap rather than a semispherical shape. Such a shape eases formation of even a metal such as titanium, which is difficult to process.

Although the vibration member 2 is suspended by using the pole 7 and the ceiling 8 as vibration member supporters in the embodiment, a pyramid frame 207 constituted by eight pipe-shaped titanium frames 207a, which are metal frames, may be used as a vibration member supporter. The use of the pyramid frame 207 causes resonance of the titanium frames 207a so that the skull 51 and the whole body can be appropriately vibrated more effectively.

For example, the vibration generators 203 of piezoelectric devices may be disposed at the center of the titanium frames 207a. In such a case, the pyramid frame 207 is vibrated so that resonance occurs in the entire frame 207. Locations on the pyramid frame 207 to which the vibration generators 203 are attached are not specifically limited. As illustrated in FIG. 7, a user may perform Zen meditation or lie down on the bone vibration sensing device 101. By appropriately vibrating the whole body in the manner described above, meditation effects can be enhanced.

### Other Embodiments

The embodiment described above may have the following configuration.

Although the vibration member 2 has the spherical shape in the embodiment, the vibration member 2 may have a dome shape whose cross-sectional shape is an elongated circle or an oval, as well as a perfect semispherical shape, in terms of limitations of manufacturing, for example. The vibration member 2 may have a cap shape as described in the variation, and is not limited to a specific shape as long as the vibration member 2 covers a skull with a predetermined spacing left between the vibration member 2 and the skull.

The foregoing embodiments are merely preferred examples in nature, and are not intended to limit the disclosure, applications, and use of the application.

What is claimed is:

1. A skull vibration device comprising:
an electrical signal generator configured to generate an electrical signal;
an amplifier configured to amplify the electrical signal from the electrical signal generator;
a vibration generator configured to convert the electrical signal transmitted from the amplifier to a mechanical vibration;
a metal vibration member to which a vibration generating portion of the vibration generator is directly coupled, and which is configured to cover a skull of a human body with a predetermined spacing left between the vibration member and the skull; and
a vibration member supporter configured to suspend the vibration member while preventing direct or indirect contact of the vibration member with the human body, and to keep the vibration member at a predetermined distance from the skull, wherein
a vibration generated from the vibration member is transferred to the skull through a vibration of air.

2. The skull vibration device according to claim 1, wherein
the vibration member is made of a titanium semispherical plate member.

3. The skull vibration device according to claim 2, wherein
the vibration member is made of a crystallized titanium semispherical plate member subjected to anodic oxidation.

4. The skull vibration device according to claim 1, wherein
the vibration member has a through hole for suspension.

5. The skull vibration device according to claim 1, wherein
the vibration generator is constituted by one or more piezoelectric devices attached to the vibration member.

6. A method for using a skull vibration device, the skull vibration device including
an electrical signal generator configured to generate an electrical signal,
an amplifier configured to amplify the electrical signal from the electrical signal generator,
a vibration generator configured to convert the electrical signal transmitted from the amplifier to a mechanical vibration,
a metal vibration member to which a vibration generating portion of the vibration generator is directly coupled, and which is configured to cover a skull of a human body with a predetermined spacing left between the vibration member and the skull, and
a vibration member supporter configured to suspend the vibration member while preventing direct or indirect contact of the vibration member with the human body, and to keep the vibration member with the predetermined spacing left between the vibration member and the skull, the method comprising the steps of:
hanging the skull vibration device from a ceiling or a suspension member;
allowing a person to stand or sit while a skull of the person is located inside the vibration member with a predetermined spacing left between the skull and the vibration member; and
transmitting an electrical signal from the electrical signal generator and causing the vibration generator to generate vibrations including a frequency at which the skull vibrates to thereby vibrate the skull.

7. The method according to claim 6, wherein
the skull vibration device further includes
a seat electrical signal generator configured to generate an electrical signal,
a seat amplifier configured to amplify the electrical signal from the seat electrical signal generator,
a seat vibration generator configured to convert the electrical signal transmitted from the seat amplifier to a mechanical vibration,
a metal seat vibration member to which a vibration generating portion of the seat vibration generator is directly coupled, and which includes a seat portion configured to contact the human body and a side portion continuous to the seat portion, and
a seat supporting member disposed at a back side of the seat portion to cover the seat vibration generator and placed on a mount surface to support the seat portion with the seat portion separated from the mount surface such that transfer of vibration energy to the mount surface is reduced, and
the method further includes the step of allowing the person to stand or sit with the skull of the person located inside the seat vibration member with the predetermined spacing left between the seat vibration member and the skull in a state where the person stands or sits on a bone vibration sensing device configured to receive a vibration from the human body that is in contact with the seat portion.

8. The method according to claim 6, further comprising the steps of:
preparing a pyramid component constituted by a metal frame as the suspension member;
connecting another vibration generator in the suspension member to the amplifier; and
allowing the person to stand or sit under the suspension member in a state where the vibration generator in the suspension member is vibrated.
